(19) United States
(12) Patent Application Publication
(10) Pub. No.: US 2025/0236572 A1
(43) Pub. Date: Jul. 24, 2025

(54) METHOD FOR SEPARATING METHANE USING HYDRATE

(71) Applicant: GWANGJU INSTITUTE OF SCIENCE AND TECHNOLOGY, Gwangju (KR)

(72) Inventors: Youngjune PARK, Gwangju (KR); Dongju SEO, Gwangju (KR)

(21) Appl. No.: 18/753,714

(22) Filed: Jun. 25, 2024

(30) Foreign Application Priority Data
Jan. 18, 2024 (KR) 10-2024-0007834

(51) Int. Cl. C07C 7/13

(52) U.S. Cl. CPC C07C 7/13

(57) ABSTRACT

Disclosed is a method for separating methane capable of selectively separating high-purity methane from natural gas, a mixture of various gases, using a solid hydrate. The method for separating methane involves preparing a solid hydrate where a thermodynamic promoter is captured in large cavities (sll-L), and small cavities (sll-S) being empty, and supplying natural gas to the solid hydrate, allowing the selective capture of methane in the small cavities (sll-S) of the solid hydrate. The methane captured using the solid hydrate is of high purity and can be utilized in various industrial fields.

### CROSS-REFERENCE TO RELATED PATENT APPLICATION

This application claims the benefit of Korean Patent Application No. 10-2024-0007834, filed on Jan. 18, 2024, in the Korean Intellectual Property Office, the disclosure of which is incorporated herein in its entirety by reference.

### BACKGROUND OF THE INVENTIVE CONCEPT

### 1. Field of the Inventive Concept

The present inventive concept relates to a method for separating methane from natural gas, and more specifically, to a method for separating high-purity methane from natural gas using a solid hydrate.

### 2. Description of the Related Art

The concentration of carbon dioxide in the atmosphere is rapidly increasing due to industrialization, and greenhouse effects caused by carbon dioxide and resulting in global warming is accelerating, leading to the occurrence of abnormal climates such as El Niño. The seriousness of global warming and abnormal climates has led to the formation of international consensus, resulting in significant discussions such as the Kyoto Protocol in 1997, which imposed greenhouse gas reduction obligations on advanced countries, and the Paris Agreement in 2016, which included climate agreements from 195 countries. Recently, substantive discussions are underway, including the declaration of carbon neutrality by 2050 (Net-zero 2050) for reducing carbon dioxide emissions.

Natural gas is a combustible gas primarily composed of hydrocarbons and is a clean fossil fuel experiencing a surge in demand due to significantly lower carbon dioxide during combustion emissions per unit of fuel mass compared to coal and oil. Natural gas produced from oil and gas fields mainly contains methane, but also includes small amounts of impurities (such as moisture, carbon dioxide, and hydrogen sulfide), which can generate atmospheric pollutants when combusted. Therefore, it requires treatment processes such as a pretreatment process to remove impurities for the use of natural gas as an energy source, a recovery process of natural gas liquid (NGL) to recover valuable components such as ethane from natural gas, and a liquefaction process to liquefy hydrocarbons consisting primarily of methane, with small amounts of ethane and propane. Furthermore, methane, the main component of natural gas, can be used to produce useful chemicals such as hydrogen through methods including steam reforming, dry reforming, and partial oxidation, and thus there is a need for a method for separating high-purity methane from natural gas.

Meanwhile, a hydrate is a compound in which object molecules are trapped in internal cavities of the hydrogen-bonded lattice structure of water molecules. The internal cavities of hydrates can capture gases at a volume ratio of more than 150 times the volume of the hydrate itself, allowing the capture and storage of various gases such as energy gases, greenhouse gases, and natural gas. This has led to research into the utilization of gas hydrates in various industrial fields, particularly in storing natural gas in the form of hydrate. However, the separation of gases using hydrates requires high pressure and low temperature conditions for gas hydrate formation, and due to the lack of economic viability of gas hydrates themselves, the use of thermodynamic promoters is required.

Therefore, there is a need for a method for separating high-purity methane from natural gas economically.

### SUMMARY OF THE INVENTIVE CONCEPT

The present inventive concept has been made in an effort to solve the above-described problems associated with prior art, and an object of the present inventive concept is to provide a method for separating methane from natural gas with high-purity

To achieve the above-mentioned object, the present inventive concept provides a method for separating high-purity methane from natural gas by forming a solid hydrate using a thermodynamic promoter and then injecting natural gas into the solid hydrate.

The method for separating methane according to the present inventive concept involves injecting natural gas into a solid hydrate where large cavities (sll-L) are filled with a thermodynamic promoter to exclude ethane or propane and selectively capture methane within small cavities (sll-S) of the solid hydrate, allowing the separation of methane from natural gas and the recovery of high-purity methane.

### BRIEF DESCRIPTION OF THE DRAWINGS

The above and other features and advantages of the present inventive concept will become more apparent by describing in detail exemplary embodiments thereof with reference to the attached drawings in which:

FIG. 1 is a schematic diagram illustrating a method for separating methane using a solid hydrate of the present inventive concept;

FIG. 2 is a graph illustrating the methane capture behavior of solid hydrates prepared using a thermodynamic promoter of the present inventive concept, as determined by ¹³C solid-state MAS NMR;

FIG. 3 is a graph illustrating the occupancy rate of gases trapped in the cavities of the solid hydrates prepared using the thermodynamic promoter of the present inventive concept;

FIG. 4 depicts 3D graphs illustrating the gases and thermodynamic promoters trapped in the cavities of the liquid hydrates prepared using the thermodynamic promoter, as determined by in-situ Raman spectroscopy;

FIG. 5 depicts 2D graphs illustrating the gases trapped in the cavities of the liquid hydrates prepared using the thermodynamic promoter, as determined by in-situ Raman spectroscopy; and

FIG. 6 is a graph illustrating the comparison of the purity and uptake of methane separated using the solid hydrates of the present inventive concept and the liquid hydrates.

### DETAILED DESCRIPTION OF THE INVENTIVE CONCEPT

Hereinafter, preferred embodiments of the present disclosure will be described in detail with reference to the accompanying drawings so that those skilled in the art can easily carry out the present inventive concept. The embodiments of the present inventive concept are provided to more completely describe the inventive concept to those skilled in the art. Thus, the embodiments of the present inventive concept can be modified in various forms, and the scope of the present inventive concept is not limited to the embodiments described below, but can be realized in other forms.

Throughout the specification of the present inventive concept, when it is said that a certain part “includes” a certain component, it means that it may further include other components without excluding other components unless otherwise stated.

As used herein, the term “sll-type hydrate” refers to a hydrate composed of 8 large cavities and 16 small cavities.

As used herein, the term “sll-S” refers to the small cavities (sll small cages) of the sll-type hydrate.

As used herein, the “sll-L” refers to the large cavities (sll large cages) of the sll-type hydrate.

### Examples

The present inventive concept provides a method for separating methane contained in natural gas using a solid hydrate. The solid hydrate may be an sll-type solid hydrate composed of 8 large cavities (sll-L) and 16 small cavities (sll-S). Specifically, the solid hydrate may contain the large cavities (sll-L) captured a thermodynamic promoter, and the small cavities (sll-S) being empty.

The method for separating methane may be preparing a solid hydrate where a thermodynamic promoter is captured in the large cavities (sll-L), and the small cavities (sll-S) being empty, and supplying natural gas containing methane, ethane, propane, etc. to the solid hydrate, allowing the selective capture of methane in the small cavities (sll-S) of the solid hydrate.

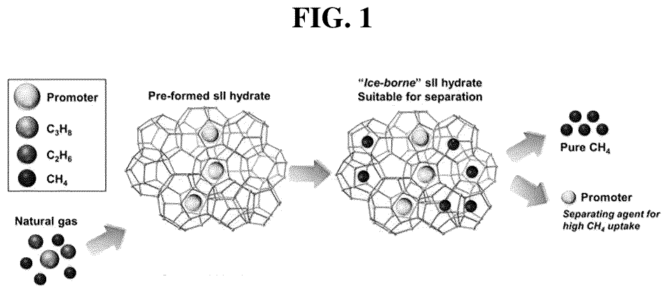

FIG. 1 is a schematic diagram illustrating the method for separating methane using the solid hydrate of the present inventive concept. Referring to FIG. 1, as a result of supplying a ternary gas mixture (CH₄+C₂H₆+C₃H₈) to the solid hydrate of the present inventive concept, methane (CH₄) is captured in the small cavities (sll-S) of the solid hydrate, allowing the selective capture of methane from the ternary gas mixture.

The solid hydrate where a thermodynamic promoter is captured in the large cavities (sll-L), with the small cavities (sll-S) being empty may be prepared by dissolving the thermodynamic promoter in a solvent to prepare a thermodynamic promoter solution, and then freezing the thermodynamic promoter solution to prepare an sll-type hydrate, followed by pulverization.

The solvent in which the thermodynamic promoter is dissolved may be water.

The thermodynamic promoter solution may be frozen at 245 K to 265 K, and specifically at 250 K to 260 K, to prepare the sll-type hydrate. The sll-type hydrate prepared by freezing the thermodynamic promoter solution may be pulverized using a mortar or sieve, specifically to sizes of 150 μm or less, preferably to sizes of 50 μm to 150 μm, to prepare the solid hydrate. Moreover, the residual gases may be removed by injecting a ternary gas mixture (CH₄+C₂H₆+C₃H₈) into the pulverized solid hydrated. Specifically, the residual gases may be removed by injecting the ternary gas mixture three or more times, and preferably the residual gases may be removed by injecting the ternary gas mixture three to five times.

The thermodynamic promoter solution may contain the thermodynamic promoter in an amount ranging from 4 mol % to 6 mol %, specifically ranging from 5 mol % to 6 mol %. Preferably the thermodynamic promoter solution may contain the thermodynamic promoter in an amount of 5.6 mol %.

The thermodynamic promoter may be an sll hydrate former, and specifically may comprise at least one compound selected from the group consisting of tetrahydrofuran (THF), cyclopentane (CP), 1,2-epoxycyclopentane (ECP), trimethylene oxide (TMO), 1,4-dioxane, 1,3-dioxolane (DIOX), methyl-cyclohexane, acetone, and tert-butyl amine. Preferably, the thermodynamic promoter may comprise at least one compound selected from the group consisting of tetrahydrofuran (THF), trimethylene oxide (TMO), and 1,3-dioxolane (DIOX).

The natural gas may be a mixed gas containing methane (CH₄), ethane (C₂H₆), and propane (CH₈). Specifically, the natural gas may be a mixed gas containing 90% methane, 7% ethane, and 3% propane. The ethane and propane are not captured by the thermodynamic promoter trapped in the large cavities (sll-L) of the solid hydrate, while the methane is captured in the small cavities (sll-S) of the solid hydrate, allowing the selective capture of methane from the natural gas, which is the mixed gas.

The methane separated from natural gas by the solid hydrate may have a purity of more than 95%, specifically more than 99%, preferably more than 99.5%.

The methane separated from natural gas by the solid hydrate may have an uptake of more than 50 v/v, specifically ranging from 50 v/v to 85 v/v, preferably ranging from 50 v/v to 81 v/v.

Hereinafter, the present inventive concept will be described in detail through Preparation Examples and Experimental Examples.

### Preparation Example 1: Preparation of Solid Hydrates Using Ice-Borne Method

### 1-1. Preparation of Solid Hydrate Using THE

5.6 mol % of tetrahydrofuran (THF) which is a stoichiometric concentration capable of forming sll hydrate was stored in a freezer maintained at 254 K. The frozen solution of sll hydrate formed in the freezer was pulverized to less than 150 μm using a mortar and sieve in a liquid nitrogen environment.

The pulverized hydrate sample was put into a reactor cooled with liquid nitrogen and then transferred to a water-ethanol bath set at a temperature of 268 K. Subsequently, the residual gases in the reactor were removed by injecting a pre-cooled ternary gas mixture (CH₄+C₂H₆+C₃H₈) three times, thereby preparing a solid hydrate (THF-hydrate) using THF.

### 1-2. Preparation of Solid Hydrate Using TMO

Except for using 5.6 mol % of trimethylene oxide (TMO) instead of THF, a solid hydrate (TMO-hydrate) using TMO was prepared in the same manner as in Preparation Example 1-1.

### 1-3. Preparation of Solid Hydrate Using DIOX

Except for using 5.6 mol % of 1,3-dioxolane (DIOX) instead of THF, a solid hydrate (DIOX-hydrate) using DIOX was prepared in the same manner as in Preparation Example 1-1.

### Preparation Example 2: Gas Separation Using Solid Hydrate

### 2-1. Gas Separation Using THF-Hydrate

The THF-hydrate prepared in Preparation Example 1-1 was reacted with a ternary gas mixture (CH₄+C₂H₆+C₃H₈) pressurized to 1 MPa for 2 weeks to separate the ternary gas mixture.

### 2-2. Gas Separation Using TMO-Hydrate

Except for using TMO-hydrate instead of THF-hydrate, the ternary gas mixture was separated in the same manner as in Preparation Example 2-1.

### 2-2. Gas Separation Using DIOX-Hydrate

Except for using DIOX-hydrate instead of THF-hydrate, the ternary gas mixture was separated in the same manner as in Preparation Example 2-1.

### Comparative Example 1: Gas Separation Using Pure-Hydrate

The ternary gas mixture (CH₄+C₂H₆+C₃H₈) was put into a reactor set at 268 K to prepare a pure-hydrate.

The prepared pure-hydrate was reacted with a ternary gas mixture (CH₄+C₂H₆+C₃H₈) pressurized to 1.5 MPa for 2 weeks in a reactor set at 268 K to separate the ternary gas mixture.

### Comparative Example 2: Gas Separation Using Liquid Hydrate Prepared by Water-Borne Method

### 2-1. Preparation of THE Liquid Hydrate and Gas Separation Using the Same

5.6 mol % of THF which is a stoichiometric concentration capable of forming sll hydrate was put into a reactor set at 285 K to prepare a liquid hydrate (W-THF-hydrate) using THF.

The prepared W-THF-hydrate was reacted with a ternary gas mixture (CH₄+C₂H₆+C₃H₈) pressurized to 2.4 MPa for 2 weeks in a reactor set at 285 K to separate the ternary gas mixture.

### 2-2. Preparation of TMO Liquid Hydrate and Gas Separation Using the Same

Except for using 5.6 mol % of trimethylene oxide (TMO) instead of THE, a liquid hydrate (W-TMO-hydrate) using TMO was prepared in the same manner as in Comparative Example 2-1.

The prepared W-THF-hydrate was reacted with a ternary gas mixture (CH₄+C₂H₆+C₃H₈) pressurized to 2.1 MPa for 2 weeks in a reactor set at 273 K to separate the ternary gas mixture.

### 2-3. Preparation of Liquid Hydrate Using DIOX and Gas Separation Using the Same

Except for using 5.6 mol % of 1,3-dioxolane (DIOX) instead of THF, a liquid hydrate (W-DIOX-hydrate) using DIOX was prepared in the same manner as in Comparative Example 2-1.

The prepared W-DIOX-hydrate was reacted with a ternary gas mixture (CH₄+C₂H₆+C₃H₈) pressurized to 2.1 MPa for 2 weeks in a reactor set at 277 K to separate the ternary gas mixture.

### Experimental Example 1: Identification of Gas Capture Behavior

### 1-1. Identification of Gas Capture Behavior of Solid Hydrates

The THF-hydrate from Preparation Example 2-1, the TMO-hydrate from Preparation Example 2-2, the DIOX-hydrate from Preparation Example 2, and the pure-hydrate from Comparative Example 1 reacted with the ternary gas mixture were analyzed by ¹³C solid-state MAS NMR to identify the captured gas.

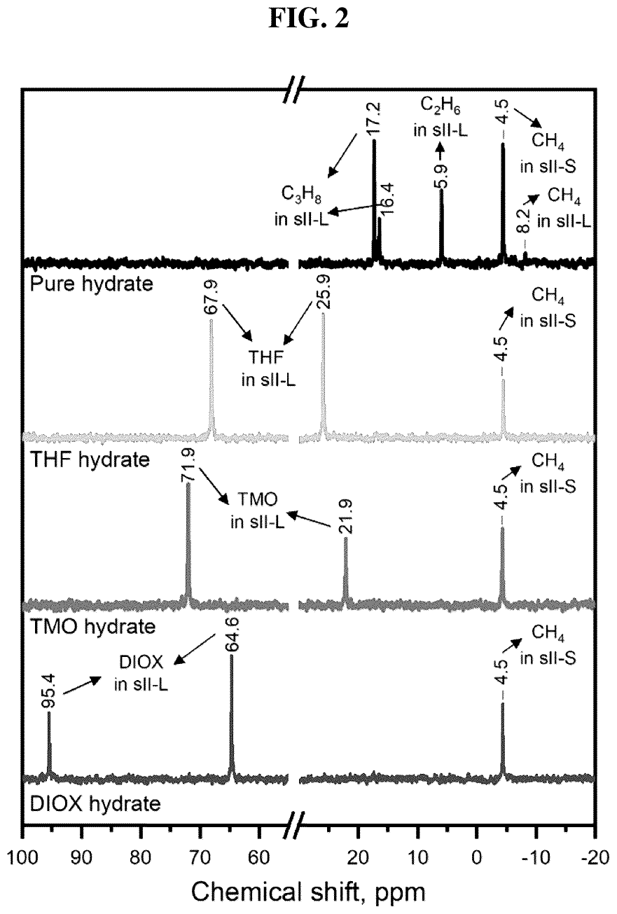

FIG. 2 is a graph illustrating the methane capture behavior of the solid hydrates prepared using the thermodynamic promoter of the present inventive concept, as determined by ¹³C solid-state MAS NMR. Referring to FIG. 2, it can be seen that in the case of the pure-hydrate, methane (CH₄) was captured in the small cavities (sll-S), while ethane (C₂H₆) and propane (C₃H₈) were captured in the large cavities (sll-L).

On the contrary, it can be seen that in the case of the THF-hydrate from Preparation Example 2-1, THF occupied the sll-L, preventing the capture of ethane and propane, and only methane was captured in sll-S. Moreover, it can be seen that in the case of the TMO-hydrate from Preparation Example 2-2, TMO occupied the sll-L, also preventing the capture of ethane and propane, and only methane was captured in sll-S. Furthermore, it can be seen that in the case of the DIOX-hydrate from Preparation Example 2-3, DIOX occupied the sll-L, preventing the capture of ethane and propane, and only methane was captured in sll-S.

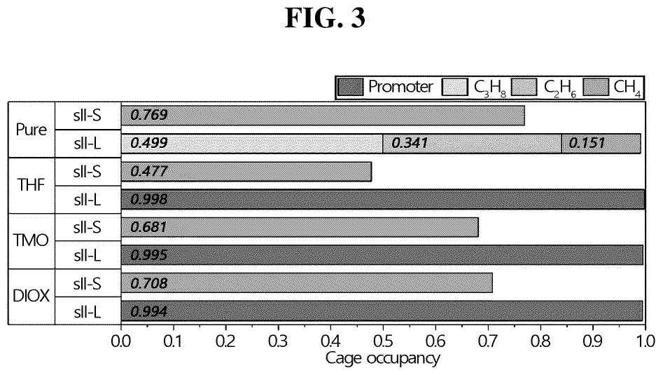

FIG. 3 is a graph illustrating the occupancy rate of gases trapped in the cavities of the solid hydrates prepared using the thermodynamic promoter of the present inventive concept. Referring to FIG. 3, as a result of comparing the occupancy rates of gases captured in sll-S and sll-L of the pure-hydrate, THF-hydrate, TMO-hydrate, and DIOX-hydrate, it can be seen that in the case of the pure-hydrate, methane, ethane, and propane were captured in sll-L. On the contrary, it can be seen that in the case of the THF-hydrate, TMO-hydrate, and DIOX-hydrate, more than 99% of THE, TMO, and DIOX were captured in sll-L, respectively, and only methane was captured in sll-S, with an occupancy rate of more than 45%.

### 1-2. Identification of Gas Capture Behavior of Liquid Hydrates

The THF-hydrate from Comparative Example 2-1, the TMO-hydrate from Comparative Example 2-2, and the DIOX-hydrate from Comparative Example 2-3 reacted with the ternary gas mixture were analyzed by in-situ Raman spectroscopy to identify the captured gas.

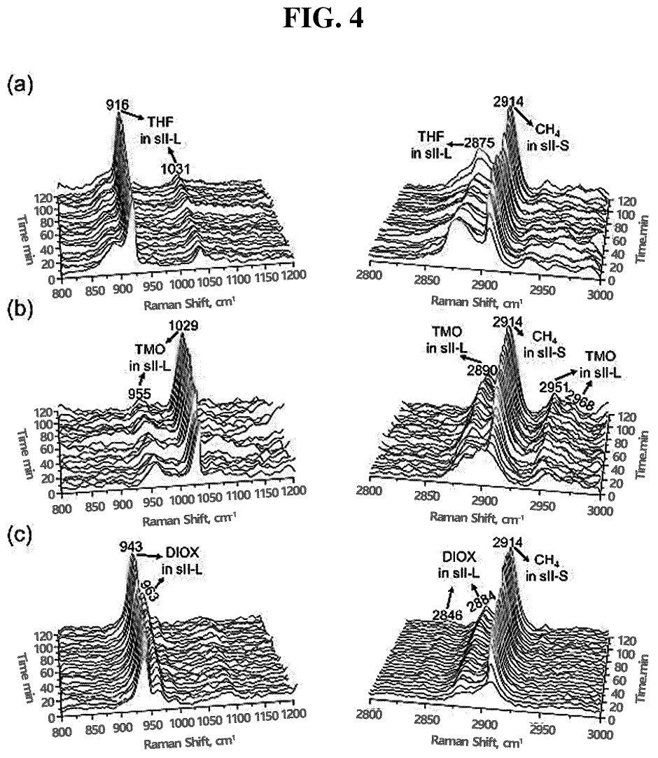

FIG. 4 depicts 3D graphs illustrating the gases trapped in the cavities of the liquid hydrates prepared using the thermodynamic promoter, as determined by in-situ Raman spectroscopy. Referring to FIG. 4 (a), it can be seen that in the case of the W-THF-hydrate, methane (CH₄) was captured in the small cavities (sll-S), while THF was captured in the large cavities (sll-L). Moreover, referring to FIG. 4 (b), it can be seen that in the W-TMO-hydrate, methane (CH₄) was captured in sll-S, while TMO was captured in sll-L. Furthermore, referring to FIG. 4 (c), it can be seen that in the case of the W-DIOX-hydrate, methane (CH₄) was captured in sll-S, while DIOX was captured in sll-L.

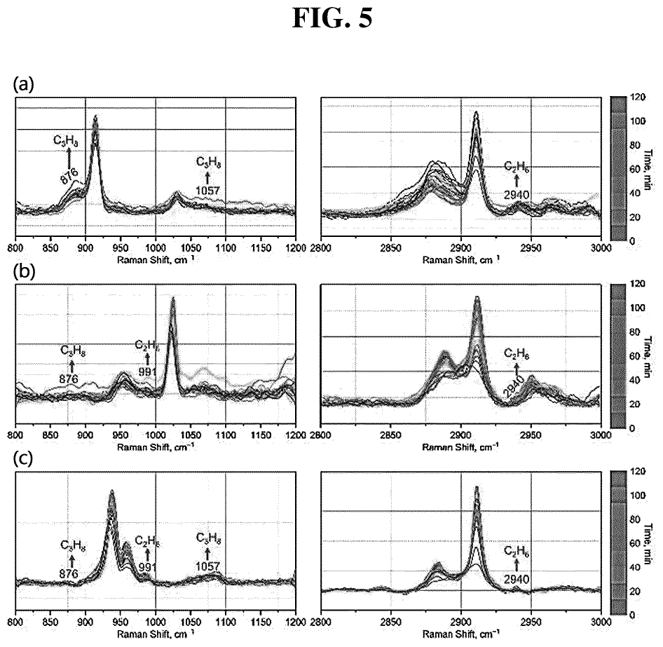

FIG. 5 depicts 2D graphs illustrating the gases trapped in the cavities of the liquid hydrates prepared using the thermodynamic promoter, as determined by in-situ Raman spectroscopy. Referring to FIG. 5 (a), it can be seen that in the case of the W-THF-hydrate, not only THF but also ethane and propane were captured in sll-L. Moreover, referring to FIG. 5 (b), it can be seen that in the case of the W-TMO-hydrate, not only TMO but also ethane and propane were captured in sll-L. Furthermore, referring to FIG. 5 (c), it can be seen that in the case of the W-DIOX-hydrate, not only DIOX but also ethane and propane were captured in sll-L.

### Experimental Example 2: Comparison of Methane Purity and Uptake

The purity and uptake of methane (CH₄) captured in the solid hydrates (THF-hydrate, TMO-hydrate, and DIOX-hydrate) from Preparation Example 2 and methane caped in the liquid hydrates (W-THF-hydrate, W-TMO-hydrate, and W-DIOX-hydrate) from Comparative Example 2 were compared.

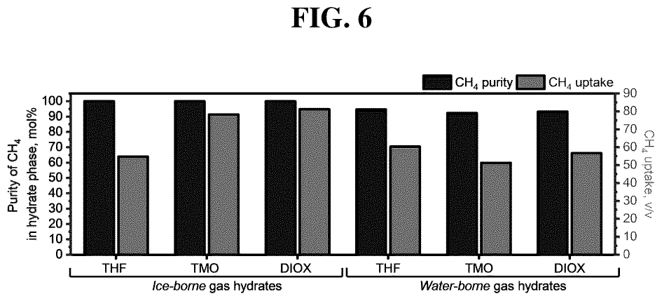

FIG. 6 is a graph illustrating the comparison of the purity and uptake of methane separated using the solid hydrates of the present inventive concept and the liquid hydrates. Referring to FIG. 6, it can be seen that the purity of methane separated using THF-hydrate, TMO-hydrate, and DIOX-hydrate is more than 99%, while the purity of methane separated using W-THF-hydrate, W-TMO-hydrate, and W-DIOX-hydrate is within the range from 90% to 95%, confirming the superior purity of methane separated using the solid hydrates.

Furthermore, referring to FIG. 6, it can be observed that the uptake of methane separated using THF-hydrate, TMO-hydrate, and DIOX-hydrate is significantly superior to that of methane separated using TMO-hydrate and DIOX-hydrate.

In conclusion, the present inventive concept has prepared the solid hydrates, THF-hydrate, TMO-hydrate, and DIOX-hydrate, using the thermodynamic promoter, tetrahydrofuran (THF), trimethylene oxide (TMO), or 1,3-dioxolane (DIOX), through an ice-borne method.

Moreover, as a result of identifying the gas capture behavior of the solid hydrates, THF-hydrate, TMO-hydrate, and DIOX-hydrate, of the present inventive concept, it was confirmed that the thermodynamic promoter was previously captured in the large cavities (sll-L), allowing only methane to be captured in the small cavities (sll-S) of the solid hydrates.

Furthermore, as a result of identifying the purity of methane separated using the solid hydrates, THF-hydrate, TMO-hydrate, and DIOX-hydrate, of the present inventive concept, it was confirmed that the purity of the methane was more than 99%.

While the inventive concept has been shown and described with reference to certain preferred embodiments thereof, it will be understood by those skilled in the art that various changes in form and details may be made therein without departing from the spirit and scope of the inventive concept as defined by the appended claims. Therefore, the scope of the inventive concept is defined not by the detailed description of the inventive concept but by the appended claims, and all differences within the scope will be construed as being included in the present inventive concept.

What is claimed is:

1. A method for separating methane, the method comprising:
preparing a solid hydrate where a thermodynamic promoter is captured in large cavities (sll-L), with small cavities (sll-S) being empty;
supplying natural gas to the solid hydrate; and
selectively capturing methane in the small cavities (sll-S) of the solid hydrate.

2. The method for separating methane of claim 1, wherein preparing the solid hydrate comprises:
dissolving the thermodynamic promoter in a solvent to prepare a thermodynamic promoter solution;
freezing the thermodynamic promoter solution to prepare a frozen product; and
pulverizing the frozen product.

3. The method for separating methane of claim 2, wherein the thermodynamic promoter solution contains the thermodynamic promoter in an amount ranging from 4 mol % to 6 mol %.

4. The method for separating methane of claim 2, wherein the solvent is water.

5. The method for separating methane of claim 1, wherein the thermodynamic promoter comprises at least one compound selected from the group consisting of tetrahydrofuran (THF), cyclopentane (CP), 1,2-epoxycyclopentane (ECP), trimethylene oxide (TMO), 1,4-dioxane, 1,3-dioxolane (DIOX), methyl-cyclohexane, acetone, and tert-butyl amine.

6. The method for separating methane of claim 1, wherein the natural gas contains methane (CH₄), ethane (C₂H₆), and propane (C₃H₈).

7. The method for separating methane of claim 1, wherein the methane captured in the small cavities (sll-S) of the solid hydrate has a purity of more than 99%,

8. The method for separating methane of claim 1, wherein the methane captured in the small cavities (sll-S) of the solid hydrate has an occupancy rate ranging from 50% to 70%.
